(19) United States
(12) Patent Application Publication
(10) Pub. No.: US 2023/0096277 A1
(43) Pub. Date: Mar. 30, 2023

(54) CATHETER PUMP MOTOR ASSEMBLY INCLUDING LUBRICATED ROTOR CHAMBER

(71) Applicant: TC1 LLC, St. Paul, MN (US)

(72) Inventors: Sarah E. Van Keulen, San Jose, CA (US); Ted Y Su, Sunnyvale, CA (US); Phyllis K. Yuen, Fremont, CA (US); Stephen D. Pacetti, San Jose, CA (US)

(21) Appl. No.: 17/823,389

(22) Filed: Aug. 30, 2022

Related U.S. Application Data
(60) Provisional application No. 63/250,656, filed on Sep. 30, 2021.

(51) Int. Cl. A61M 60/414; A61M 60/13; A61M 60/174; A61M 60/216; F16C 19/18

(52) U.S. Cl. CPC A61M 60/414; A61M 60/13; A61M 60/174; A61M 60/216; F16C 19/18

(57) ABSTRACT

A catheter pump system includes a shaft assembly, an impeller coupled to a distal portion of the shaft assembly, and a motor assembly coupled to a proximal portion of the shaft assembly. The motor assembly is configured to drive the impeller via the shaft assembly. The motor assembly includes a rotor, a stator disposed radially outward of the rotor, and a rotor chamber disposed radially between the stator and the rotor. The rotor chamber at least partially encloses the rotor. The rotor chamber is at least partially filled with a lubricant to reduce a friction of the rotor during rotation thereof.

### BACKGROUND

### Field of Invention

This application is generally directed to catheter pumps for mechanical circulatory support of a heart.

### Description of the Related Art

Heart disease is a major health problem that has high mortality rate. Physicians increasingly use mechanical circulatory support systems for treating heart failure. The treatment of acute heart failure requires a device that can provide support to the patient quickly. Physicians desire treatment options that can be deployed quickly and minimally-invasively.

Mechanical circulatory support (MCS) systems and ventricular assist devices (VADs) have gained greater acceptance for the treatment of acute heart failure such as acute myocardial infarction (MI) or to support a patient during high risk percutaneous coronary intervention (PCI). An example of an MCS system is a rotary blood pump placed percutaneously, e.g., via a catheter.

In a conventional approach, a blood pump is inserted into the body and connected to the cardiovascular system, for example, to the left ventricle and the ascending aorta to assist the pumping function of the heart. Such permanently implanted pumps are known as left ventricular assist devices (LVADs). Other known applications include placing the pump in the descending aorta, a peripheral artery, and the like. Typically, acute circulatory support devices are used to reduce the afterload on the heart muscle and provide blood flow for a period of time to stabilize the patient prior to heart transplant or for continuing support.

There is a need for improved mechanical circulatory support devices for treating acute heart failure. There is a need for minimally invasive devices designed to provide near full heart flow rate.

There is a need for a blood pump with improved performance and clinical outcomes. There is a need for a pump that can provide elevated flow rates with reduced risk of hemolysis and thrombosis. There is a need for a pump that can be inserted minimally-invasively and provide sufficient flow rates for various indications while reducing the risk of major adverse events.

In one aspect, there is a need for a heart pump that can be placed minimally-invasively, for example, through an 18FR, 14FR, or 8FR incision. In one aspect, there is a need for a heart pump that can provide an average flow rate of 4 LPM or more during operation, for example, against a backpressure of 62 mmHg of aortic pressure.

While the flow rate of a rotary blood pump can be increased by rotating the impeller faster, higher rotational speeds are known to increase the risk of hemolysis, which can lead to adverse outcomes and in some cases death. Higher speeds also lead to performance and patient comfort challenges from the rotating components. Many percutaneous ventricular assist devices (VADs) have driveshafts between the motor and impeller rotating at high speeds. Some percutaneous VADs are designed to rotate at speeds of more than 15,000 RPM, and in some cases more than 25,000 RPM in operation. The vibration, noise, and heat from the motor and driveshaft can cause discomfort to the patient, especially when positioned inside or on the body. Moreover, fluids (such as saline and/or blood) may enter the motor, which can damage the motor and/or impair operation of the catheter pump. Accordingly, there is a need for a device that improves performance and patient comfort with a high-speed motor.

There is a need for a motor configured to drive an operative device, such as an impeller, an atherectomy device, and/or another rotating feature. There is a need for an improved motor having a lubricated and/or liquid cooled rotor and/or rotor chamber. There is a need for a motor capable of rotating at relatively high speeds. These and other problems are overcome by present disclosure.

### SUMMARY

In one aspect, a catheter pump system is described. The catheter pump system includes a shaft assembly, an impeller coupled to a distal portion of the shaft assembly, and a motor assembly coupled to a proximal portion of the shaft assembly. The motor assembly is configured to drive the impeller via the shaft assembly. The motor assembly includes a rotor, a stator disposed radially outward of the rotor, and a rotor chamber disposed radially between the stator and the rotor. The rotor chamber at least partially encloses the rotor. The rotor chamber is at least partially filled with a lubricant to reduce a friction of the rotor during rotation thereof.

In another aspect, a motor assembly for a catheter pump system is described. The motor assembly includes a rotor, a stator disposed radially outward of the rotor, and a rotor chamber disposed radially between the stator and the rotor. The rotor chamber at least partially encloses the rotor. The rotor chamber is at least partially filled with a lubricant to reduce a friction of the rotor during rotation thereof.

In yet another aspect, a method for manufacturing a catheter pump system is described. The method includes providing a rotor. The rotor is configured to couple to a proximal portion of an output shaft. The method also includes coupling a stator at least partially about the rotor. The stator and the rotor define a radial gap therebetween. The method also includes coupling a rotor chamber at least partially between the stator and the rotor within the radial gap. The rotor chamber at least partially encloses the rotor. The method also includes filling the rotor chamber with a lubricant to prime the catheter pump system with the lubricant prior to operation.

### BRIEF DESCRIPTION OF THE DRAWINGS

A more complete appreciation of the subject matter of this application and the various advantages thereof can be realized by reference to the following detailed description, in which reference is made to the accompanying drawings in which:

FIG. 1A illustrates one embodiment of a catheter pump system with an impeller assembly configured for percutaneous application and operation.

FIG. 1B is a schematic view of at least one embodiment of a catheter pump system adapted to be used in the manner illustrated in FIG. 1A.

FIG. 1C is a schematic view of at least one embodiment of a catheter pump system.

FIG. 2 is a side plan view of at least one embodiment of the motor assembly of the catheter pump system shown in FIG. 1B, according to various embodiments.

FIG. 3 is a perspective exploded view of at least one embodiment of the motor assembly shown in FIG. 2.

FIG. 4 is a side cross-sectional view of at least one embodiment of the motor assembly shown in FIGS. 2-3, in which a rotor chamber of the motor assembly is filled with a lubricant, and in which a priming tube is positioned proximally of the rotor chamber.

FIG. 5 is a side cross-sectional view of at least one embodiment of the motor assembly shown in FIGS. 2-3, in which a rotor chamber of the assembly is filled with a lubricant, and in which a priming tube is positioned distally of the rotor chamber.

FIG. 6 is a perspective view of at least one embodiment of a seal for use with the motor assembly shown in FIGS. 2-5.

FIG. 7 is a side perspective view of at least one embodiment of the motor assembly shown in FIGS. 2-3, in which the motor assembly includes a plurality of ball bearing assemblies.

FIG. 8 is a perspective view of at least one embodiment of a ball bearing assembly for use with the motor assembly shown in FIGS. 2-3 and FIG. 7.

FIG. 9 is a side cross-sectional view of at least one embodiment of the motor assembly shown in FIGS. 2-3, in which a rotor chamber of the motor assembly is filled with a lubricant, and in which a center lumen is also filled with the lubricant.

FIG. 10 is a side perspective view of at least one embodiment of the motor assembly shown in FIGS. 2-3 and FIG. 9.

FIG. 11 is a side cross-sectional view of a portion of a catheter body of the motor assembly shown in FIGS. 2-3 and FIGS. 9-10, in which the catheter body is filled with a lubricant.

More detailed descriptions of various embodiments of components for heart pumps useful to treat patients experiencing cardiac stress, including acute heart failure, are set forth below.

### DETAILED DESCRIPTION

Embodiments of a catheter pump system are described herein. In some embodiments, the catheter pump system includes a motor having a rotor coupled to an output shaft defining a center lumen therethrough and a stator assembly surrounding the rotor. A flow diverter, which may include one or more chambers, such as a rotor chamber, may be disposed or coupled between the stator and the rotor, and which may at least partially surround or enclose the rotor. In some embodiments, the rotor chamber may be filled with a lubricant, such as a gel or oil, to lubricate the rotor during operation. In some embodiments, the lubricant may also be provided within the center lumen defined by the output shaft and/or a lumen of a catheter body that is in fluid communication with the center lumen of the output shaft, such as to lubricate a drive shaft disposed within the catheter body. In some embodiments, a flow of fluid, such as saline, may be provided to components, such as an impeller assembly, located distally of the motor. In some embodiments, no saline returns proximally through the motor. In some embodiments, saline may return proximally through the center lumen of the output shaft, which may not be filled with lubricant in at least these embodiments. In some embodiments, different types of bearings, such as journal bearings and/or ball bearings, may be disposed relative to the motor to support the output shaft. In some embodiments, one or more seals, such as oil bath seals, may be provided to fluidly isolate the motor from blood and/or fluid, such as blood and fluid returning or leaking proximally and/or distally toward rotating portions of the motor. In some embodiments, one or more septa may be provided, such as within the drive shaft, within the output shaft, and the like, to further limit or prevent blood and/or fluid entering the motor. In some embodiments, one or more anticoagulant agents, anti-foaming agents, and/or hydrophilic agents may be added to system surfaces, such as to reduce or eliminating blood clotting, thrombogenicity, and the like.

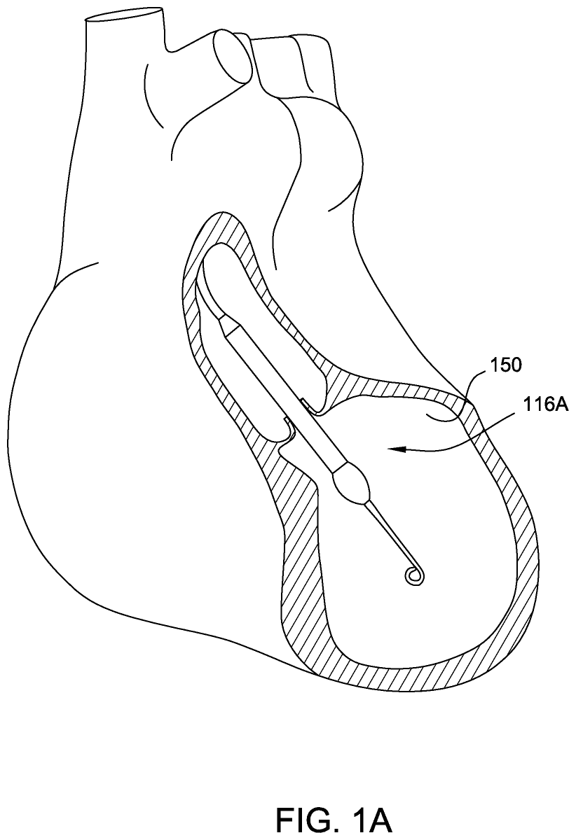

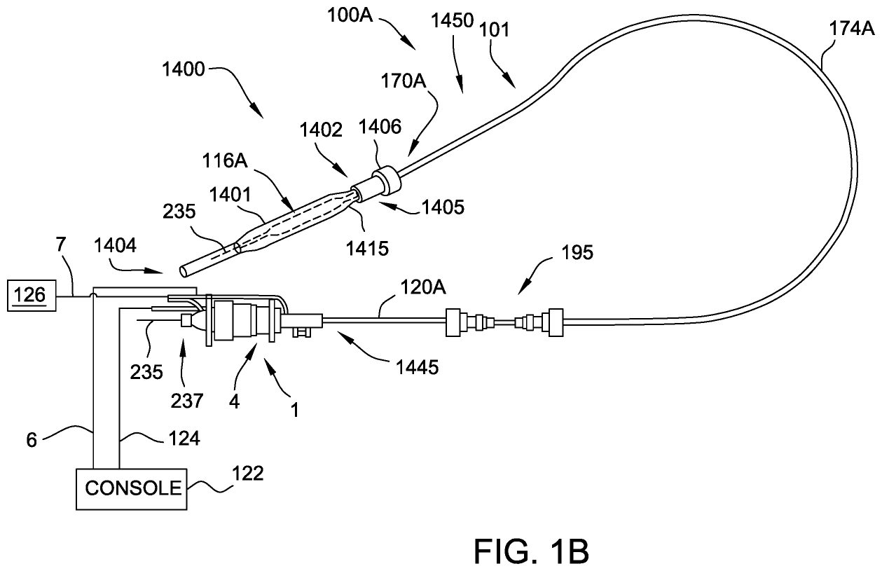

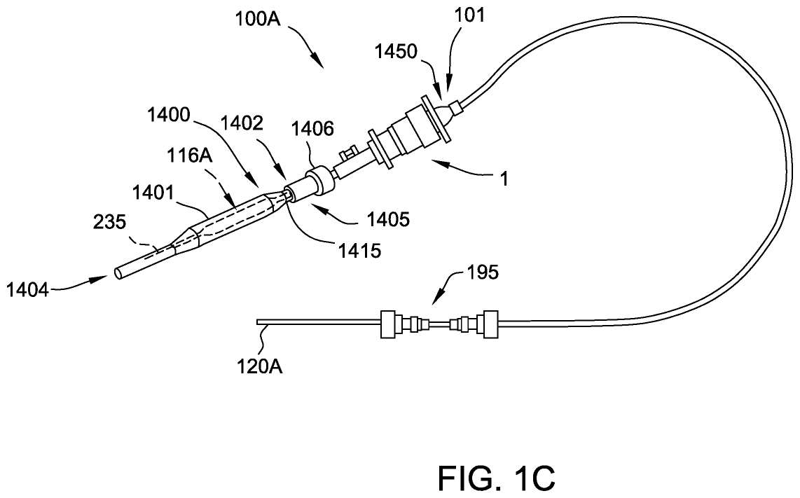

FIGS. 1A-1B show aspects of an exemplary catheter pump 100A that can provide relatively high blood flow rates (i.e. full or near full blood flow). As shown in FIG. 1B, the pump 100A includes a motor assembly 1 driven by a console 122, which can include an electronic controller and various fluid handling systems. The console 122 directs the operation of the motor 1 and an infusion system that supplies a flow of fluid in the pump 100A. Additional details regarding the exemplary console 122 may be understood from U.S. Patent Publication No. US 2014/0275725, the contents of which are incorporated by reference herein in their entirety and for all purposes.

The pump 100A includes a catheter assembly 101 that can be coupled with the motor assembly 1 and can house an impeller in an impeller assembly 116A within a distal portion of the catheter assembly 101 of the pump 100A. In various embodiments, the impeller is rotated remotely by the motor 1 when the pump 100A is operating. For example, the motor 1 can be disposed outside the patient. In some embodiments, the motor 1 is separate from the console 122, e.g., to be placed closer to the patient. In the exemplary system the pump is placed in the patient in a sterile environment and the console is outside the sterile environment. In at least one embodiment, the motor is disposed on the sterile side of the system. In other embodiments, the motor 1 is part of the console 122.

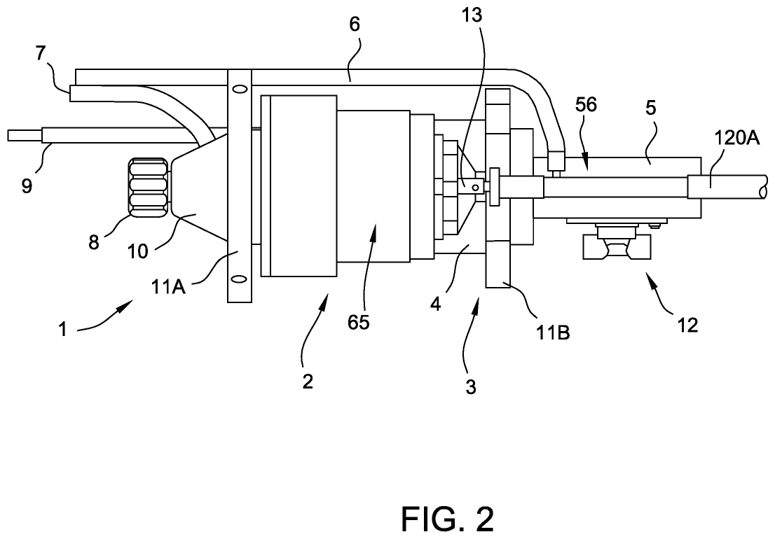

In some embodiments, the motor 1 is miniaturized to be insertable into the patient. For example, FIG. 1C is a schematic view of at least one embodiment of a catheter pump system. FIG. 1C is similar to FIG. 1B, except the motor 1 is miniaturized for insertion into the body. As shown in FIG. 1C, for example, the motor 1 can be disposed proximal the impeller assembly 116A. The motor 1 can be generally similar to the motor assembly shown in FIG. 2, except the motor 1 is sized and shaped to be inserted into the patient's vasculature. One or more electrical lines may extend from the motor to the console outside the patient. The electrical lines can send signals for controlling the operation of the motor. Additionally or alternatively, in some embodiments, the electrical lines may also provide electrical power to the motor. Such embodiments allow a drive shaft coupled with the impeller and disposed within the catheter assembly 101 to be much shorter, e.g., shorter than the distance from the aortic valve to the aortic arch (about 5 cm or less). Various embodiments of the motor assembly 1 are disclosed herein, including embodiments having a rotor disposed within a stator assembly. In some embodiments, the housing in which the motor 1 of FIG. 1C is disposed can be sealed from fluids (e.g., blood and/or saline) so as to isolate the electrical lines from the fluids.

FIG. 1A illustrates one use of the catheter pump 100A. A distal portion of the pump 100A including a catheter assembly including the impeller assembly 116A is placed in the left ventricle (LV) 150 of the heart to pump blood from the LV 150 into the aorta. The pump 100A can be used in this way to treat a wide range of heart failure patient populations including, but not limited to, cardiogenic shock (such as acute myocardial infarction, acute decompensated heart failure, or postcardiotomy), myocarditis, and others.

The pump can also be used for various other indications including to support a patient during a cardiac intervention such as a high-risk percutaneous coronary intervention (PCI) or ablation. One convenient manner of placement of the distal portion of the pump 100A in the heart is by percutaneous access and delivery using a modified Seldinger technique or other methods familiar to cardiologists. These approaches enable the pump 100A to be used in emergency medicine, a catheter lab and in other medical settings.

Modifications can also enable the pump 100A to support the right side of the heart. Example modifications that could be used for right side support include providing delivery features and/or shaping a distal portion that is to be placed through at least one heart valve from the venous side, such as is discussed in U.S. Pat. Nos. 6,544,216; 7,070,555; and US 2012-0203056A1, all of which are hereby incorporated by reference herein in their entirety for all purposes.

The impeller assembly 116A (e.g., the impeller and cannula) can be expandable and collapsible. In the collapsed state, the distal end of the catheter pump 100A can be advanced to the heart, for example, through an artery. In the expanded state the impeller assembly 116A is able to pump blood at relatively high flow rates. In particular, the expandable cannula and impeller configuration allows for decoupling of the insertion size and flow rate. In other words, it allows for higher flow rates than would be possible through a lumen limited to the insertion size with all other things being equal.

In FIGS. 1A and 1B, the impeller assembly 116A is illustrated in the expanded state. The collapsed state can be provided by advancing a distal end 170A of an elongate body 174A distally over the impeller assembly 116A to cause the impeller assembly 116A to collapse. This provides an outer profile throughout the catheter assembly and catheter pump 100A that is of small diameter during insertion, for example, to a catheter size of about 12.5 FR in various arrangements. In other embodiments, the impeller assembly 116A is not expandable.

The mechanical components rotatably supporting the impeller within the impeller assembly 116A permit relatively high rotational speeds while providing low friction, and controlling heat and particle generation that can come with high speeds. In some embodiments, the infusion system delivers a cooling and lubricating solution to the distal portion of the catheter pump 100A for these purposes. The space for delivery of this fluid is extremely limited. Providing secure connection and reliable routing of fluid into and out of the catheter pump 100A is critical and challenging in view of the small profile of the catheter assembly 101.

When activated, the catheter pump 100A can effectively support, restore and/or increase the flow of blood out of the heart and through the patient's vascular system. In various embodiments disclosed herein, the pump 100A can be configured to produce a maximum flow rate (e.g. zero mm Hg backpressure) of greater than 4 LPM, greater than 4.5 Lpm, greater than 5 Lpm, greater than 5.5 Lpm, greater than 6 Lpm, greater than 6.5 Lpm, greater than 7 Lpm, greater than 7.5 Lpm, greater than 8 Lpm, greater than 9 Lpm, or greater than 10 Lpm. In various embodiments, the pump 100A can be configured to produce an average flow rate at 62 mmHg backpressure of greater than 2 Lpm, greater than 2.5 Lpm, greater than 3 Lpm, greater than 3.5 Lpm, greater than 4 Lpm, greater than 4.25 Lpm, greater than 4.5 Lpm, greater than 5 Lpm, greater than 5.5 Lpm, greater than 6 Lpm, greater than 6.5 Lpm, greater than 7 Lpm, greater than 8 Lpm, or greater than 9 Lpm.

Various aspects of the pump and associated components can be combined with or substituted for those disclosed in U.S. Pat. Nos. 7,393,181; 8,376,707; 7,841,976; 7,022,100; and 7,998,054, and in U.S. Pub. Nos. 2011/0004046; 2012/0178986; 2012/0172655; 2012/0178985; and 2012/0004495, the entire contents of each of which are incorporated herein for all purposes by reference. In addition, various aspects of the pump and system can be combined with those disclosed in U.S. Patent Publication No. US 2013/0303970, entitled “DISTAL BEARING SUPPORT,” filed on Mar. 13, 2013; U.S. Patent Publication No. US 2014/0275725, entitled “FLUID HANDLING SYSTEM,” filed on Mar. 11, 2014; U.S. Patent Publication No. US 2013/0303969, entitled “SHEATH SYSTEM FOR CATHETER PUMP,” filed on Mar. 13, 2013; U.S. Patent Publication No. US 2013/0303830, entitled “IMPELLER FOR CATHETER PUMP,” filed on Mar. 13, 2013; U.S. Patent Publication No. US 2014/0012065, entitled “CATHETER PUMP,” filed on Mar. 13, 2013; and U.S. Patent Publication No. US 2014/0010686, entitled “MOTOR ASSEMBLY FOR CATHETER PUMP,” filed on Mar. 13, 2013, the entire contents of each of which are incorporated herein for all purposes by reference.

Moving from a distal end 1450 of the catheter assembly 101 of the catheter pump 100A of FIG. 1B to a proximal end 1455, a priming apparatus 1400 can be disposed over the impeller assembly 116A. As explained above, the impeller assembly 116A can include an expandable cannula or housing and an impeller with one or more blades. As the impeller rotates, blood can be pumped proximally (or distally in some implementations) to function as a cardiac assist device.

In various embodiments, the pump is configured to be primed with fluid. Turning to FIG. 1B, a priming apparatus 1400 can be disposed over the impeller assembly 116A near the distal end portion 170A of the elongate body 174A. The priming apparatus 1400 can be used in connection with a procedure to expel air from the impeller assembly 116A, e.g., any air that is trapped within the housing or that remains within the elongate body 174A near the distal end 170A. For example, the priming procedure may be performed before the pump is inserted into the patient's vascular system, so that air bubbles are not allowed to enter and/or injure the patient. The priming apparatus 1400 can include a primer housing 1401 configured to be disposed around both the elongate body 174A and the impeller assembly 116A.

A sealing cap 1406 can be applied to the proximal end 1402 of the primer housing 1401 to substantially seal the priming apparatus 1400 for priming, i.e., so that air does not proximally enter the elongate body 174A and also so that priming fluid does not flow out of the proximal end of the housing 1401. In some embodiments, sealing cap 1406 may be a rotating hemostatic valve (“RHV”), which may, for example, seal primer housing 1401 onto outer sheath 174A. The sealing cap 1406 can couple to the primer housing 1401 in any way known to a skilled artisan. In some embodiments, the sealing cap 1406 is threaded onto the primer housing by way of a threaded connector 1405 located at the proximal end 1402 of the primer housing 1401. The sealing cap 1406 can include a sealing recess disposed at the distal end of the sealing cap 1406. The sealing recess can be configured to allow the elongate body 174A to pass through the sealing cap 1406.

The priming operation can proceed by introducing fluid into the sealed priming apparatus 1400 to expel air from the impeller assembly 116A and the elongate body 174A. Fluid can be introduced into the priming apparatus 1400 in a variety of ways. For example, fluid can be introduced distally through the elongate body 174A into the priming apparatus 1400. In other embodiments, an inlet, such as a luer, can optionally be formed on a side of the primer housing 1401 to allow for introduction of fluid into the priming apparatus 1400. A gas permeable membrane can be disposed on a distal end 1404 of the primer housing 1401. The gas permeable membrane can permit air to escape from the primer housing 1401 during priming.

The priming apparatus 1400 also can advantageously be configured to collapse an expandable portion of the catheter pump 100A. The primer housing 1401 can include a funnel 1415 where the inner diameter of the housing 1401 decreases from distal to proximal. The funnel 1415 may be gently curved such that relative proximal movement of impeller assembly 116A (e.g., an impeller housing thereof) causes impeller assembly 116A to be collapsed by the funnel 1415.

During or after the impeller assembly 116A has been fully collapsed, the distal end 170A of the elongate body 174A can be moved distally relative to the collapsed impeller assembly 116A. After the impeller assembly 116A is fully collapsed and retracted into the elongate body 174A, the catheter pump 100A can be removed from the priming apparatus 1400 before a percutaneous heart procedure is performed, e.g., before the pump 100A is activated to pump blood. In at least one embodiment, a catheter body 120A can pass within the elongate body 174A, such that the external elongate body 174A can axially translate relative to the internal catheter body 120A. In addition, as described in additional detail herein and in at least some embodiments, a fluid (e.g., a lubricant, saline, and the like) may be transported within catheter body 120A and provided to impeller assembly 116A.

The embodiments disclosed herein may be implemented such that the total time for infusing, priming, or flushing the system is minimized or reduced. As used herein, the terms “infusing, priming, and flushing” may be generally used interchangeably to refer to the process of introducing fluid within primer housing 1401, as described herein. For example, in some implementations, the time to fully infuse the system can be about six minutes or less. In other implementations, the time to infuse can be about three minutes or less. In yet other implementations, the total time to infuse the system can be about 45 seconds or less. It should be appreciated that lower times to infuse can be advantageous for use with cardiovascular patients. Although the described pump is primed with fluid, one will appreciate from the description herein that the priming may be optional. For example, the pump can be prepared such that all air is removed before it is packaged. In another example, air is removed by placing the pump under vacuum.

With continued reference to FIG. 1B, the elongate body 174A extends from the impeller assembly 116A in a proximal direction to an intermediate portion, which as used herein, may be referred to as a fluid supply device 195, such as for example, to refer to the capability of fluid supply device 195 to transport fluid, as described herein, from console 122 toward impeller assembly 116A and/or proximally in a return flow path from impeller assembly 116A. The fluid supply device 195 is configured to allow for fluid to enter the catheter assembly 101 of the catheter pump 100A and/or for waste fluid to leave the catheter assembly 101 of the catheter pump 100A.

A catheter body 120A (which may be referred to as an “inner sheath” within elongate body 174A and which also passes through the elongate body 174A) can extend proximally and couple to the motor assembly 1. As discussed in more detail herein, the motor assembly 1 can provide torque to a drive shaft that extends from the motor assembly 1 through the catheter body 120A to couple to an impeller shaft at or proximal to the impeller assembly 116A. As described herein, the catheter body 120A can pass within the elongate body 174A such that the external elongate body 174A can axially translate relative to the internal catheter body 120A.

Further, as shown in FIG. 1B, a fluid supply line 6 can fluidly couple with the console 122 to supply saline or other fluid to the catheter pump 100A. The saline or other fluid can pass through an internal lumen of the internal catheter body 120A and can provide lubrication to the impeller assembly 116A and/or chemicals to the patient. The supplied fluid (e.g., saline, dextrose, glucose solution, heparin, or infusate) can be supplied to the patient by way of the catheter body 120A at any suitable flow rate. For example, in various embodiments, the fluid is supplied to the patient at a flow rate in a range of 15 mL/hr to 50 mL/hr, or more particularly, in a range of 20 mL/hr to 40 mL/hr, or more particularly, in a range of 25 mL/hr to 35 mL/hr. One or more electrical conduits 124 can provide electrical communication between the console 122 and the motor assembly 1. A controller within the console 122 can control the operation of the motor assembly 1 during use.

Fluid (e.g., saline) can be provided from outside the patient (e.g., by way of one or more supply bags) to the pump through a supply lumen in the catheter body. The fluid can return to the motor assembly 1 by way of a lumen (e.g., a central or interior lumen) of the catheter body. For example, as explained herein, the fluid can return to the motor assembly 1 through the same lumen in which the drive shaft is disposed. In addition, a waste line 7 can extend from the motor assembly 1 to a waste reservoir 126. Waste fluid from the catheter pump 100A can pass through the motor assembly 1 and out to the reservoir 126 by way of the waste line 7.

In various embodiments, the waste fluid flows to the motor assembly 1 and the reservoir 126 at a flow rate which is lower than that at which the fluid is supplied to the patient. For example, some of the supplied fluid may flow out of the catheter body 120A and into the patient by way of one or more bearings. The waste fluid (e.g., a portion of the fluid which passes proximally back through the motor from the patient) may flow through the motor assembly 1 at any suitable flow rate, e.g., at a flow rate in a range of 5 mL/hr to 20 mL/hr, or more particularly, in a range of 10 mL/hr to 15 mL/hr. Although described in terms of fluid and waste lines, one will appreciate that the pump and motor be configured to operate without fluid flushing. One purpose of the fluid supply is to cool the motor. In the case of a micromotor dimensioned and configured to be inserted percutaneously, there may not be a need for fluid cooling because the motor heat will be dissipated by the body.

With continuing reference to FIG. 1B, access can be provided to a proximal end of the catheter assembly 101 of the catheter pump 100A prior to or during use. In one configuration, the catheter assembly 101 may be delivered over a guidewire 235. For example, in at least some embodiments, the guidewire 235 may be conveniently extended through the entire length of the catheter assembly 101 of the catheter pump 100A and out of a proximal end 1455 of the catheter assembly 101, such as after catheter pump 100A is primed as described herein. Specifically, in at least one embodiment, guidewire 235 may be extended through catheter assembly 101 after priming of impeller assembly 116A and sheathed or re-sheathed within elongate body 174A. In various embodiments, the connection between the motor assembly 1 and the catheter assembly 101 is configured to be permanent, such that the catheter pump, the motor housing and the motor are disposable components. However, in other implementations, the coupling between the motor housing and the catheter assembly 101 is disengageable, such that the motor and motor housing can be decoupled from the catheter assembly 101 after use. In such embodiments, the catheter assembly 101 distal of the motor can be disposable, and the motor and motor housing can be re-usable.

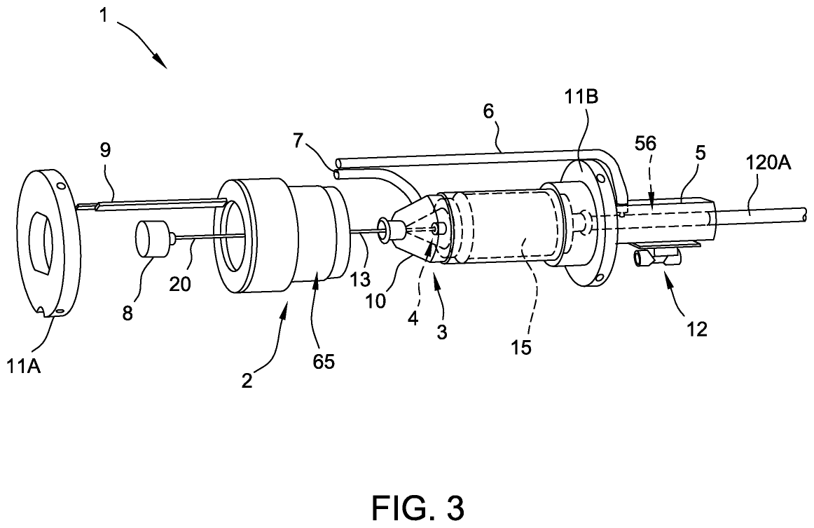

In addition, FIG. 1B illustrates the guidewire 235 extending from a proximal guidewire opening 237 in the motor assembly 1. Before inserting the catheter assembly 101 of the catheter pump 100A into a patient, a clinician may insert the guidewire 235 through the patient's vascular system to the heart to prepare a path for the impeller assembly 116A to the heart. In some embodiments, the catheter pump 100A can include a guidewire guide tube 20 (see FIG. 3) passing through a central internal lumen of the catheter pump 100A from the proximal guidewire opening 237. The guidewire guide tube 20 can be pre-installed in the catheter pump 100A to provide the clinician with a preformed pathway along which to insert the guidewire 235.

In one approach, the guidewire 235 is placed into a peripheral blood vessel, and along the path between that blood vessel and the heart and into a heart chamber, e.g., into the left ventricle. Thereafter, a distal end opening of the catheter pump 100A and guidewire guide tube 20 can be advanced over the proximal end of the guidewire 235 to enable delivery of the catheter pump 100A. After the proximal end of the guidewire 235 is delivered proximally within the catheter pump 100A and emerges from the guidewire opening 237 and/or guidewire guide tube 20, the catheter pump 100A can be advanced into the patient. In one method, the guidewire guide tube 20 is withdrawn proximally while holding the catheter pump 100A.

Alternatively, the clinician can insert the guidewire 235 through the proximal guidewire opening 237 and urge the guidewire 235 along the guidewire guide tube. The clinician can continue urging the guidewire 235 through the patient's vascular system until the distal end of the guidewire 235 is positioned in the desired position, e.g., in a chamber of the patient's heart, a major blood vessel or other source of blood. As shown in FIG. 1B, a proximal end portion of the guidewire 235 can extend from the proximal guidewire opening 237. Once the distal end of the guidewire 235 is positioned in the heart, the clinician can maneuver the impeller assembly 116A over the guidewire 235 until the impeller assembly 116A reaches the distal end of the guidewire 235 in the heart, blood vessel or other source of blood. The clinician can remove the guidewire 235 and the guidewire guide tube. The guidewire guide tube can also be removed before or after the guidewire 235 is removed in some implementations. After removing at least the guidewire 235, the clinician can activate the motor 1 to rotate the impeller and begin operation of the pump 100A.

In yet another embodiment, catheter pump 100A is configured to be inserted using a modified Seldinger technique. The pump may be configured with a lumen therethrough for receiving a guidewire. Unlike the embodiment described above, however, the guidewire is threaded through the pump without a guidewire guide tube. One will appreciate from the description herein that other configurations may be employed for loading the pump onto a guidewire and/or moving the pump to the target location in the body. Examples of similar techniques are described in U.S. Pat. No. 7,022,100 and U.S. Pub. No. 2005/0113631, the entire contents of which patent and publication are incorporated herein for all purposes.

FIGS. 2 and 3 further illustrate aspects of embodiments of the motor assembly 1 shown in FIG. 1B. In FIG. 2, the motor assembly 1 is shown in an assembled state. In FIG. 3, the motor assembly 1 is shown in an exploded view. Accordingly, the motor assembly 1 can include a stator assembly 2 and a rotor 15 disposed radially within the stator assembly 2. The motor assembly 1 also includes a flow diverter 3, which can be configured as a manifold for directing fluid through one or more passages in the catheter pump 100A. In some cases, the flow diverter 3 is at least partially disposed radially between the stator assembly 2 and the rotor 15 (FIGS. 2-3). The flow diverter 3 can be fluidly sealed about the rotor 15 and a proximal portion 56 of the catheter body 120A. The seal prevents leakage and also can prevent the fluid from contacting the stator assembly 2.

The flow diverter 3 can include a distal chamber 5 within which the proximal portion 56 of the catheter body 120A is disposed and a rotor chamber 4 within which the rotor 15 is disposed. The distal chamber 5 is fluidly connected with the catheter. The rotor chamber 4 is fluidly connected with the waste line 7. The flow diverter 3 can also have a proximal chamber 10 in some embodiments. Where provided, the distal chamber 5, rotor chamber 4, and proximal chamber 10 can be in fluid communication within the flow diverter 3.

One or more flanges 11A, 11B can mechanically couple the flow diverter 3 to an external housing (not shown). The flanges 11A, 11B are examples of mount structures that can be provided, which can include in various embodiments dampers to isolate the motor assembly 1 from external shock or vibration. In some embodiments, mount structures can include dampers configured to isolate an outer housing or the environment external to the motor assembly 1 from shock or vibration generated by the motor assembly 1.

Further, an optional pressure sensor assembly 12 is configured to measure the pressure at a distal portion of the catheter pump 100A by, for example, measuring the pressure of a column of fluid that extends distally through a lumen of the catheter body 120A. In addition, the guidewire guide tube 20 can extend proximally through the motor assembly 1 and can terminate at a tube end cap 8 (FIG. 3). As explained above, the guidewire 235 can be inserted within the guide tube 20 for guiding the catheter pump 100A to the heart.

In various embodiments, the rotor 15 and stator assembly 2 are configured as or are components of a “frameless-style” motor for driving the impeller assembly 116A at the distal end of the pump 100A. For example, the stator assembly 2 can comprise a stator and a plurality of conductive windings producing a controlled magnetic field. The windings can be wrapped about or in a stationary portion 65 of the stator assembly 2. The rotor 15 can comprise a magnetic material, e.g., can include one or more permanent magnets.

In some embodiments, the rotor 15 can comprise a multi-pole magnet, e.g., a four-pole or six-pole magnet. Providing changing electrical currents through the windings of the stator assembly 2 can create magnetic fields that interact with the rotor 15 to cause the rotor 15 to rotate. This is commonly referred to as commutation. The console 122 can provide electrical power (e.g., 24V) to the stator assembly 2 to drive the motor assembly 1. One or more leads 9 can electrically communicate with the stator assembly 2, e.g., with one or more Hall sensors used to detect the speed and/or position of the motor. In other embodiments, other sensors (e.g., optical sensors) can be used to measure motor speed.

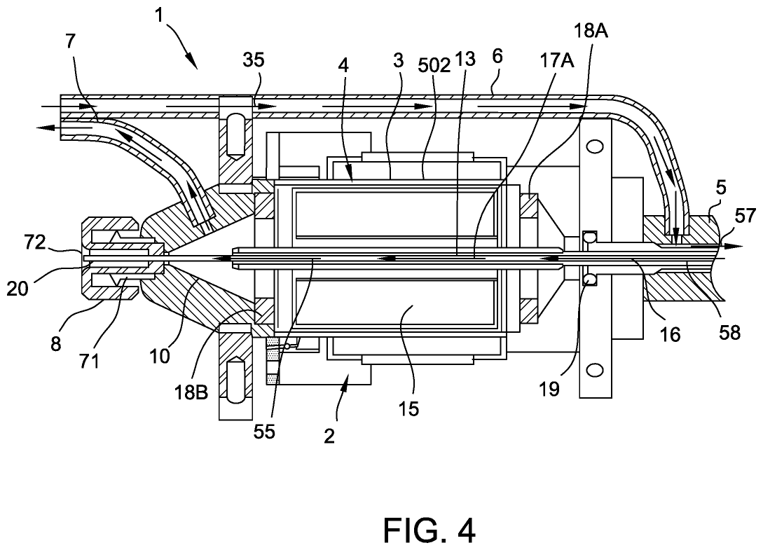

The rotor 15 can be secured to an output shaft 13 (which can comprise a hollow shaft with a central lumen) such that rotation of the rotor 15 causes the output shaft 13 to rotate. In various embodiments, the motor assembly 1 can comprise a direct current (DC) brushless motor. In other embodiments, other types of motors can be used, such as AC motors, gearhead motor, etc. As shown in FIG. 4, first and second journal bearings 18A, 18B can be provided about the output shaft 13 to radially and/or longitudinally center the output shaft 13 and thereby the rotor 15 relative to the stator assembly 2.

FIG. 4 shows that the output shaft 13 (which is secured to the rotor 15) can be mechanically coupled with the proximal end portion of a drive shaft 16. The drive shaft 16 extends distally through an internal lumen of the catheter body 120A. A distal end portion of the drive shaft 16 is mechanically connected with the impeller. Thus, rotation of the rotor 15 causes the output shaft 13 to rotate, which, in turn, causes the drive shaft 16 and the impeller to rotate. FIG. 4 also shows that a lumen 55 can extend through the output shaft 13 and the rotor 15. In certain embodiments, the lumen 55 is coupled with a lumen of the catheter body 120A such that the guidewire guide tube 20 can extend through the lumen 55 within the rotor 15 and into the lumen of the catheter body 120A. In addition, the drive shaft 16 comprises a braided shaft having an internal lumen.

FIG. 4 shows the tube end cap 8 welded or otherwise secured to a proximal end portion of the guide tube 20. The cap 8 can be removably engaged (e.g., screwed or otherwise removably locked) over a female receiver 71 that is secured in a proximal end of the proximal chamber 10. For example, the proximal end of the female receiver 71 can be disposed in a counterbore of the cap 8, while the guide tube 20 extends through the central opening of the cap 8. In a locked configuration, one or more tabs of the receiver 71 can be rotated such that the tab(s) slide under a corresponding tab in the counterbore of the cap 8. In an unlocked configuration, the tab(s) of the receiver 71 can be rotated relative to the tabs of the cap 8. In the illustrated embodiment, the cap 8 can be fixed to the guide tube 20; in other embodiments, the receiver 71 can be fixed to the guide tube 20. Engaging the cap 8 to the receiver 71 can advantageously prevent the guide tube 20 from accidentally being removed from or slid within the catheter pump 100A, e.g., if the patient or clinician impacts the cap 8. To remove the guide tube 20 (e.g., in various embodiments, before, during, and/or after delivery of the impeller assembly 116A to the heart), the clinician can disengage the cap 8 from the receiver 71 and can pull the guide tube 20 from the catheter pump 100A, for example, by pulling proximally on the end cap 8.

A resealable septum 72 (e.g., a resealable closure member) can be provided at the proximal end of the flow diverter 3, e.g., near the distal end of the cap 8 when the cap 8 is in place. When the guidewire guide tube 20 is removed from the pump 100A, the septum 72 will naturally reseal the pathway proximally from the motor assembly 1 such that fluid does not exit the assembly 1. An advantage of the assembly described herein is that the cap 8 is locked and will not be dislodged without rotating and unlocking cap 8 from receiver 71. Otherwise, the cap 8 can slide axially if it is inadvertently bumped by the patient or clinician. This potentially results in the guide tube 20 being pulled out from the distal-most end of the impeller assembly 116A, and because the guide tube cannot be re-inserted, the clinician either has to use the catheter pump 100A without a guide or get a new pump.

With continued reference to FIG. 4, it can be important to ensure that the motor assembly 1 is adequately cooled. In various embodiments, it can be important to provide a heat removal system to limit buildup of heat in the motor assembly 1 during operation. For example, it can be important to maintain external surfaces of the motor assembly 1 at a temperature less than about 40° C. if the motor assembly 1 is positioned near the patient. For example, an external surface of an external housing of the motor assembly 1 may be kept at or below this temperature. In some respects, regulatory guidelines can require that no part in contact with skin exceed 40° C. To that end, various strategies for heat management are employed by the inventions described herein. It should be appreciated that, as used herein, cooling refers to transferring away or dissipating heat, and in certain respects, cooling is used interchangeably with removing heat.

Various components of the motor assembly 1 generate heat. For example, moving parts within the motor assembly 1 (e.g., the rotating output shaft 13 and/or drive shaft 16) can generate heat by virtue of losses through friction, vibrations, and the like, which may increase the overall temperature of the motor assembly 1. Further, heat can be generated by the electrical current flowing through the stator assembly 2 and/or by induction heating caused by conductive components inside a rotating magnetic field. Furthermore, friction between the bearings 18A, 18B and the output shaft 13 and/or friction between the drive shaft 16 and the inner wall of catheter body 120A may also generate undesirable heat in the motor assembly. Inadequate cooling can result in temperature increases of the motor assembly 1, which can present patient discomfort, health risks, or performance losses. This can lead to undesirable usage limitations and engineering complexity, for example, by requiring mitigation for differential heat expansion of adjacent components of different materials. Accordingly, various embodiments disclosed herein can advantageously transfer away generated heat and cool the motor assembly 1 such that the operating temperature of the assembly 1 is sufficiently low to avoid such complexities of use or operation and/or other components of the system. For example, various heat transfer components can be used to move heat away from thermal generation sources and away from the patient. Various aspects of the illustrated device herein are designed to reduce the risk of hot spots, reduce the risk of heat spikes, and/or improve heat dissipation to the environment and away from the patient.

In some embodiments, the catheter pump makes use of the fluid supply system already embedded in the pump to cool the motor assembly 1 and housing. In some embodiments, heat absorbing capacity of fluid flowing through the flow diverter 3 is used to cool the motor assembly 1. As shown in FIG. 4, the supply line 6 can supply fluid 35 from a source (e.g., a fluid bag) to an outer lumen 57 of the catheter body 120A. The supplied fluid 35 can travel distally toward the impeller assembly 116A to lubricate rotating components in the catheter assembly 101 and/or supply fluid to the patient.

A seal 19 (e.g., an O-ring) can be provided between the rotor chamber 4 and the distal chamber 5 to prevent unwanted backflow or leakage of the fluid 35 into the rotor chamber 4. In this context, backflow is flow of fluid 35 proximally into the distal chamber 5 rather than distally within the lumen 57. Such flow is to be prevented to ensure that the fluid 35 is initially exposed to moving parts in a distal portion of the catheter assembly 101 to lubricate and cool such distal components.

### EXAMPLE 1

In at least some embodiments, at least a portion 17A of fluid 35 can return proximally through an inner lumen 58 of catheter body 120A. For example, after initially cooling distal components, such as impeller assembly 116A, at least some of fluid 35 can flow proximally within lumen 58 surrounding drive shaft 16. In addition, in at least some embodiments, portion 17A of fluid 35 may flow from lumen 58 of catheter body 120A into lumen 55 of output shaft 13.

As described herein, lumen 55 and lumen 58 may also define a region through which guidewire guide tube 20 extends prior to removal of guide tube 20 during a surgical procedure. As a result, it can be seen that at least a portion 17A of fluid 35 may flow distally within lumen 57 to lubricate impeller assembly 116A and/or to supply fluids and/or medicaments (e.g., saline) to a patient during surgery. At least some portion 17A of the distally flowing fluid 35 may return, via lumen 55 of output shaft 13, to lubricate and/or cool motor 1, such as by flowing through lumen 55 to absorb and transfer heat generated by motor 1.

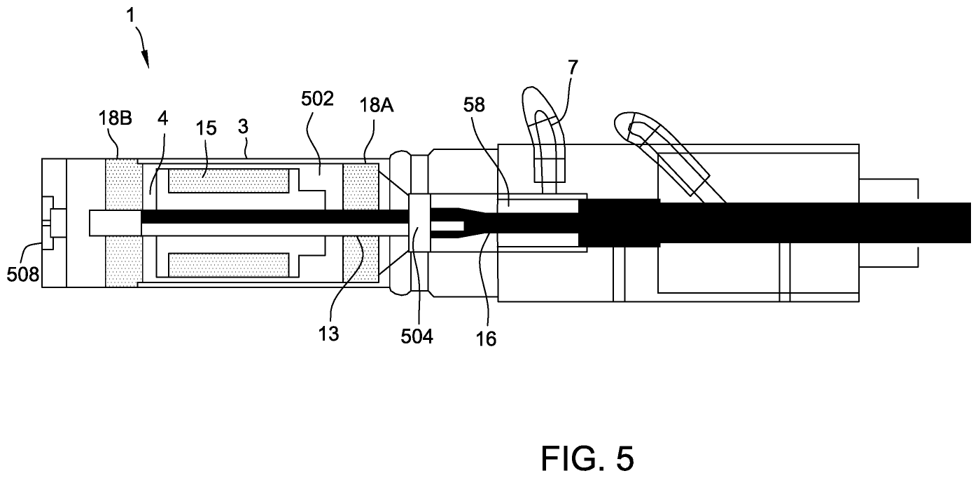

With reference to FIG. 4 and FIG. 5, in some embodiments, at least a portion of flow diverter 3 may be sealed and filled with a lubricant 502. For example, rotor chamber 4 may be sealed, as described herein, and filled with lubricant 502. Lubricant 502 may include any suitable lubricating substance or composition, such as any gel, any oil, any jelly or grease, and/or any other substance or composition that may be used to lubricate rotating components, such as rotor 15 during operation of motor 1. In some embodiments, lubricant 502 may include, but is not limited to, substances such as silicone fluid, freon, a low viscosity hydrocarbon material, such as heptane, mineral oil, vegetable oil, glycerin, water, combinations of glycerin and water, propylene glycol, combinations of propylene glycol and water, and the like.

In the example embodiment, rotor chamber 4 may be fluidly sealed using one or more annular seals, such as seal 504 (see FIG. 5). In some embodiments, seal 504 may include an oil bath seal, a gasket, an O-ring, and/or any other suitable annular seal. As shown, in at least some embodiments, seal 504 may be positioned distal to (and in contact with) journal bearing 18A. As described herein, seal 504 may be included to prevent and/or reduce a flow or proximal leakage of fluid 35 and/or blood, such that at least rotor 15 is substantially fluidly isolated from contact with proximally flowing fluid 35 and/or blood (e.g., as used herein, “blood back”).

It will be appreciated that isolation of rotor 15 (and/or other parts and portions of motor 1, such as any rotating part of motor 1) may result in the technical effect and improvement that, during operation, blood and/or fluid 35 is prevented, or substantially prevented, from entering motor 1. Reduction and/or elimination of proximally flowing fluid 35 and/or blood back may, in addition, reduce or eliminate coagulation of blood and/or fluid 35, foaming of blood and/or fluid 35, and/or other undesirable consequences of contact between blood and one or more rotating components (e.g., rotor 15, drive shaft 16, etc.) or motor 1.

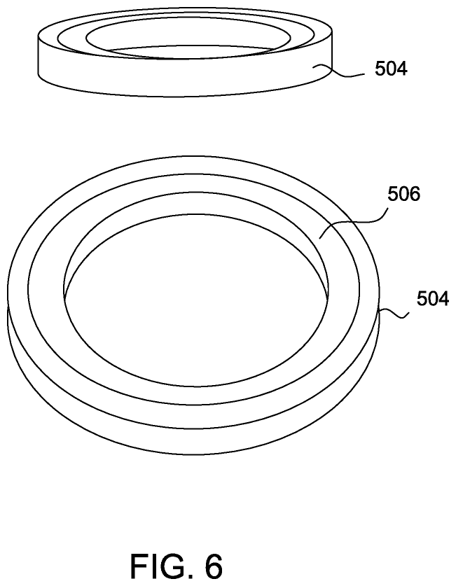

An example seal 504 is shown with reference to FIG. 6. As shown, in at least one embodiment, seal 504 may be an oil seal and/or an oil bath seal, which may be used to contain lubricant 502 within rotor chamber 4, such that lubricant 502 is prevented, or substantially prevented, from leaking from rotor chamber 4. In at least some embodiments, seal 504 may, alternatively and/or additionally, prevent or reduce proximal flow of blood and/or other fluid 35, such that blood and/or fluid 35 is prevented, or substantially prevented, from entering rotor chamber 4.

Accordingly, as shown in FIG. 6, in at least one embodiment, seal 504 may be spring-loaded (and/or, for example, include a spring-loaded portion or spring portion). In addition, seal 504 may include a synthetic (e.g., rubber) material that is resistant to degradation and/or corrosion in the presence of lubricant 502 as well as that is configured to liquidly seal rotor chamber 4 from blood back and other fluid leakage. In some embodiments, seal 504 may include an annular channel, such as channel 506, formed in one or more surfaces and configured to engage and/or otherwise seal with journal bearing 18A.

In addition to these features, as shown with continuing reference to FIG. 5, in at least some embodiments, a septum 508 may be included within output shaft 13. For example, septum 508 may be provided within an inner diameter of output shaft 13 near a proximal end thereof (e.g., proximally of rotor 15), such as for example, to prevent and/or reduce blood and or fluid 35 entering rotor chamber 4 and/or proximal chamber 10 via output shaft 13 and/or journal bearing 18B.

### EXAMPLE 2

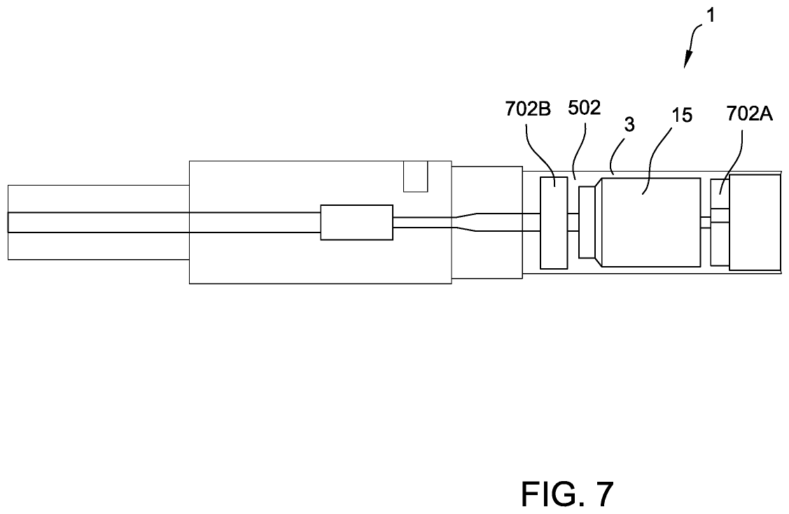

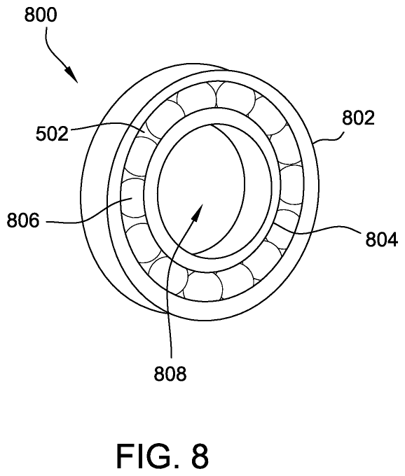

With reference to FIG. 7, in some embodiments, journal bearings 18A and 18B may be replaced with ball bearing assemblies 702A and 702B, respectively. An example ball bearing assembly 800, such as either of ball bearing assemblies 702A and/or 702B is shown in FIG. 8. Specifically, as shown, ball bearing assembly 800 may include an annular or ring-shaped outer portion 802 concentric with an annular or ring-shaped inner portion 804. A plurality of balls, such a steel balls 806, may be interposed between outer portion 802 and inner portion 804. A lubricant, such as lubricant 502, may infill between balls 806 to lubricate bearing assembly 800. Inner portion 804 may further define an annular receiving portion 808 configured to securely receive and engage a rotatable member, such as for example, output shaft 13. In some embodiments, other types of bearings may be used, such as for example, roller bearings tapered roller bearings, and the like. For example, in at least some implementations, bearings may be selected at least in part based upon a desired rotational speed of output shaft 13. For instance, in at least one embodiment, ball bearing assemblies 702A and 702B may be selected to facilitate higher rotational speeds, while other types of bearings, such as roller bearings and/or tapered roller bearings may be selected to facilitate lower rotational speeds (e.g., speeds less than those facilitated by ball bearing assemblies 702A and 702B).

As described herein, in at least some embodiments, at least a portion of flow diverter 3 may be sealed and filled with lubricant 502, such as in conjunction with using ball bearing assemblies 702A and 702B. For example, rotor chamber 4 may be sealed, as described herein, and filled with lubricant 502. In some embodiments, flow diverter 3 may be prefilled (or “primed”) with lubricant 502, such as during manufacture of motor 1. In some embodiments, flow diverter 3 may be filled or primed with lubricant 502 prior to use, such as by introducing lubricant 502 into flow diverter 3 through waste line 7.

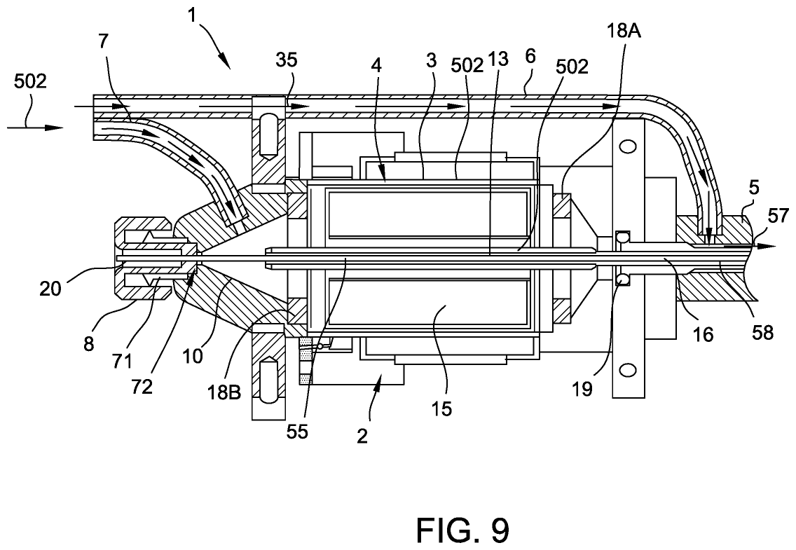

In some embodiments, waste line 7 may be positioned proximally and may feed directly into proximal chamber 10 of flow diverter 3 (e.g., see FIG. 9). In other embodiments, as shown, for example, in FIGS. 5 and 7, waste line 7 may be positioned distally of flow diverter 3 and arranged to feed lubricant 502 into flow diverter 3 through an inner lumen, such as, but not limited to, lumen 58 of catheter body 120A and/or lumen 55 of output shaft 13. In both cases, however, waste line 7 may be used to introduce lubricant, in at least some embodiments, into at least one chamber of flower diverter 3, such as rotor chamber 4, proximal chamber 10, and/or distal chamber 5. Moreover, in such embodiments, waste line 7 may not be referred to herein as a waste line, but as a “priming tube” and/or “lubricant introducer line,” as described herein.

In addition to these features, in at least some embodiments, as described herein, waste line 7 may also be used to channel waste fluid 35 returning from impeller assembly 116A out of motor 1 and into a waste reservoir 126. In some embodiments, waste line 7 may not be used to channel waste fluid 35 out of motor 1. Rather, in at least some embodiments, waste line 7 may only be used to introduce lubricant 502 into flow diverter 3, such that waste line 7 may function as a lubricant introducer line (or “priming tube”) rather than as a waste line. In addition, in at least one embodiment, an additional priming tube (not shown) may also be provided, where for example, the additional priming tube may be fluidly coupled with at least a portion of flow diverter 3.

Moreover, in at least some embodiments, output shaft 13 extends through and is mechanically coupled to rotor 15 (e.g., by welding output shaft 13 within rotor 15 and/or by any other suitable securing or fastening technique). In some embodiments, as described herein, fluid 35 may return proximally through lumen 55 of output shaft 13. For example, in at least one embodiment, rotor chamber 4 may be sealed and filled with lubricant 502, while lumen 55 may channel return flow proximally through output shaft 13 and out through waste line 7 into waste reservoir 126 (e.g., see FIG. 4).

In some embodiments, however, lumen 55 may also be filled with lubricant 502, and no return flow of fluid 35 may occur (e.g., see FIG. 9). Rather, motor 1 may not facilitate return flow of fluid 35, in at least some embodiments, because lubricant 502 may facilitate cooling (e.g., reduction of friction) as well as lubrication functionality, as described herein. In certain embodiments, lumen 55 is coupled with lumen 58 of the catheter body 120A, as described herein, and lubricant 502 fills lumen 58 (which is in fluid communication with lumen 55) to lubricate drive shaft 16 (which may in some embodiments include a drive cable, such as a braided or woven drive cable).

### EXAMPLE 3

Accordingly, with reference to FIG. 9, in some embodiments, fluid 35 does not return proximally through any portion of motor 1 and/or flow diverter 3. Specifically, in at least one embodiment, no portion of fluid 35 returns proximally through inner lumen 58 of catheter body 120A. Rather, fluid 35 is introduced distally of flow diverter 3 via supply line 6 into outer lumen 57 of catheter body 120A to cool distal components, such as impeller assembly 116A. However, in at least one embodiment, no portion (e.g., portion 17A, as shown in FIG. 4) of fluid 35 returns to waste reservoir 126 via flow diverter 103. Rather, a volume of fluid 35 being provided to impeller assembly 116A may be tailored to provide adequate cooling and/or lubrication as well as to be completely dissipated within the body of a patient during a surgical procedure (e.g., where fluid 35 includes a medical fluid, such as saline or heparinized saline).

In such embodiments, at least a portion of flow diverter 3 may be sealed and filled with lubricant 502. For example, rotor chamber 4 may be sealed, as described herein, and filled with lubricant 502. In some embodiments, flow diverter 3 may be prefilled (or “primed”) with lubricant 502, such as during manufacture of motor 1. In some embodiments, flow diverter 3 may be filled or primed with lubricant 502 prior to use, such as by introducing lubricant 502 into flow diverter 3 through waste line 7, where as described herein, waste line 7 may be used in at least some embodiments as a priming tube rather than to transport waste fluid.

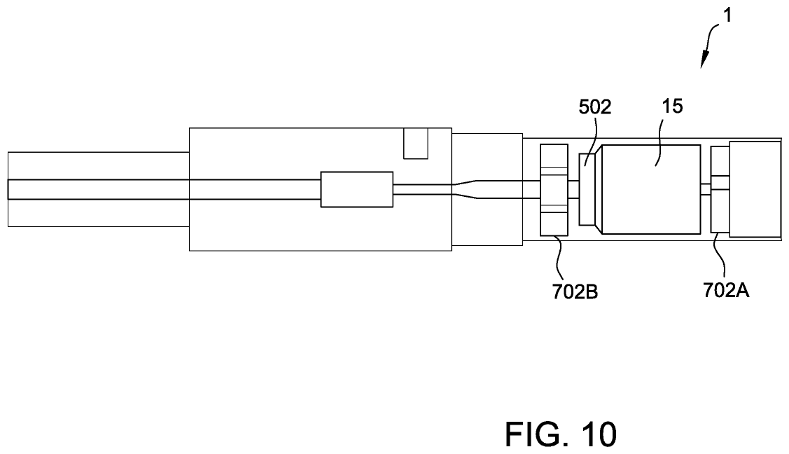

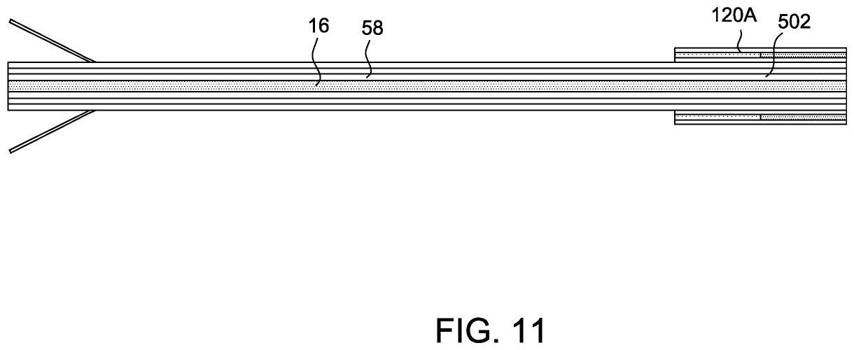

In addition to priming flow diverter 3, in some embodiments, drive shaft 16 may be lubricated, such as using lubricant 502, since it will be appreciated that in at least some embodiments, if fluid 35 is prevented from returning proximally through the inner lumen 58 of catheter body 120A, another lubrication and/or cooling technique may be desirably applied to drive shaft 16. Accordingly, in at least some embodiments, as an alternate to fluid flow returning proximally within inner lumen 58 of catheter body 120A, lubricant 502 may be applied to drive shaft 16 and/or within inner lumen 58 to lubricate drive shaft 16 therein. FIG. 10 is a perspective view of motor 1 illustrating these features. FIG. 11 is a cross-sectional view of a portion of catheter body 120A illustrating a lubricant 502 provided on drive shaft 16 within inner lumen 58 of catheter body 120A.

### EXAMPLES 4 AND 5

In some embodiments, an anticoagulant agent (such as ethylenediamine-tetraacetic acid (“EDTA”) and/or sodium heparin) may be included within flow diverter 3, such as within any of rotor chamber 4, distal chamber 5, and/or proximal chamber 10, and such as during manufacture, as described herein, and/or via waste line 7, such as in addition to lubricant 502 and/or as an alternative to lubricant 502. In some embodiments, the anticoagulant may be entrained, impregnated, otherwise included in lubricant 502.

In some embodiments, an anti-foaming agent (e.g., an oil-based, water-based, silicone-based, EO/PO-based, and/or alkyl polyacrylate-based anti-foaming agent) may be included within flow diverter 3. The anti-foaming agent may be included in addition to, or as an alternative to, the anticoagulant agent and/or lubricant 502. Thus, a variety of lubricating, anticoagulating, and/or anti-foaming formulations may be implemented within flow diverter 3. These techniques may also be applied within other lumens and device cavities, such as outer lumen 57 and/or inner lumen 58 of catheter body 120A in association with drive shaft 16.

When motor 1 is primed or otherwise filled with lubricant 502, the anticoagulating and/or anti-foaming agents described herein may dissolve into lubricant 502 (which may be water soluble, and which may include, as described herein, saline, glycerol, propylene glycol, and/or a low molecular weight poly(ethylene glycol)). Moreover, during operation, if blood comes into contact with lubricant 502 (which may be entrained or impregnated with one or more anticoagulants and/or anti-foaming agents, as described herein), lubricant 502 may reduce and/or eliminate coagulation, foaming, thrombosis, and/or other undesired consequences of contact between blood and one or more rotating components, such as drive shaft 16. As a result, motor 1 may be protected from adverse operating conditions even in the case that a small amount of blood leaks into portions of motor 1. Stated another way, these and other techniques described herein may facilitate continued operation of motor 1 in the event that some blood (or blood back) returns or leaks proximally into motor 1, and the like.

In some embodiments, one or more anticoagulant agents and/or one or more anti-foaming agents may be applied to portions of motor 1, such as surfaces of motor 1 within flow diverter 3, surfaces of drive shaft 16, surfaces of any other rotating component, and the like. By reducing the thrombogenicity of various surfaces that may be exposed to blood, coagulation of any blood that ultimately contacts these surfaces may be reduced or eliminated. Moreover, in some embodiments, and in addition to anticoagulant and/or antifoaming agents, one or more hydrophilic agents and/or substances may be added (e.g., albumin, heparin, poly(2-methoxyethylacrylate) (PMEA), polyethylene oxide (PEO), and the like). These substances may, it will be appreciated, decrease protein adhesion and clotting when blood enters portions of motor 1.

Although the embodiments disclosed herein have been described with reference to particular embodiments, it is to be understood that these embodiments are merely illustrative of the principles and applications of the present inventions. It is therefore to be understood that numerous modifications can be made to the illustrative embodiments and that other arrangements can be devised without departing from the spirit and scope of the present inventions as defined by the appended claims. Thus, it is intended that the present application cover the modifications and variations of these embodiments and their equivalents.

What is claimed is:

1. A catheter pump system comprising:
a shaft assembly;
an impeller coupled to a distal portion of the shaft assembly; and
a motor assembly coupled to a proximal portion of the shaft assembly, the motor assembly configured to drive the impeller via the shaft assembly, the motor assembly comprising:
a rotor;
a stator disposed radially outward of the rotor; and
a rotor chamber disposed radially between the stator and the rotor, the rotor chamber at least partially enclosing the rotor, the rotor chamber at least partially filled with a lubricant to reduce a friction of the rotor during rotation thereof.

2. The catheter pump system of claim 1, further comprising:
a first journal bearing positioned distally of the rotor, the first journal bearing securely receiving and supporting a first portion of the shaft assembly;
a second journal bearing positioned proximally of the rotor, the second journal bearing securely receiving and supporting a second portion of the shaft assembly; and
a first seal positioned distally of the first journal bearing and abutting the first journal bearing.

3. The catheter pump system of claim 2, wherein the first seal is an oil bath seal, and wherein the first seal includes at least a spring portion and a liquid sealing portion.

4. The catheter pump system of claim 1, further comprising a septum disposed within a proximal portion of the shaft assembly, the septum occluding the proximal portion of the shaft assembly.

5. The catheter pump system of claim 1, further comprising a priming tube fluidly coupled to the rotor chamber, the priming tube configured to receive and transfer the lubricant into the rotor chamber.

6. The catheter pump system of claim 1, further comprising:
a first ball bearing assembly positioned distally of the rotor, the first ball bearing assembly securely receiving and supporting a distal portion of the shaft assembly; and
a second ball bearing assembly positioned proximally of the rotor, the second ball bearing assembly securely receiving and supporting a proximal portion of the shaft assembly.

7. The catheter pump system of claim 6, wherein the first ball bearing assembly and the second ball bearing assembly include the lubricant, such that during operation, the first ball bearing assembly and the second ball bearing assembly are at least partially self-lubricating.

8. The catheter pump system of claim 1, wherein the shaft assembly extends through a center portion of the rotor, and wherein the shaft assembly is at least partially filled with the lubricant.

9. The catheter pump system of claim 1, further comprising a catheter body that houses a drive cable of the shaft assembly, and wherein the catheter body is at least partially filled with the lubricant.

10. The catheter pump system of claim 1, wherein at least one of the shaft assembly and the rotor chamber include a surface coating of at least one of an anticoagulant agent, an anti-foaming agent, and a hydrophilic agent.

11. A motor assembly for a catheter pump system, the motor assembly comprising:
a rotor;
a stator disposed radially outward of the rotor; and
a rotor chamber disposed radially between the stator and the rotor, the rotor chamber at least partially enclosing the rotor, the rotor chamber at least partially filled with a lubricant to reduce a friction of the rotor during rotation thereof.

12. The motor assembly of claim 11, further comprising:
a journal bearing positioned distally of the rotor, the journal bearing securely receiving and supporting a portion of a shaft assembly; and
a seal positioned distally of the journal bearing and abutting the journal bearing.

13. The motor assembly of claim 12, wherein the seal is an oil bath seal, and wherein the seal includes at least a spring portion and a liquid sealing portion.

14. The motor assembly of claim 11, further comprising a shaft assembly coupled to the rotor and a septum disposed within a proximal portion of the shaft assembly, the septum occluding the proximal portion of the shaft assembly.

15. The motor assembly of claim 11, further comprising a priming tube fluidly coupled to the rotor chamber, the priming tube configured to receive and transfer the lubricant into the rotor chamber.

16. The motor assembly of claim 11, further comprising:
a first ball bearing assembly positioned distally of the rotor, the first ball bearing assembly securely receiving and supporting a distal portion of a shaft assembly coupled to the rotor; and
a second ball bearing assembly positioned proximally of the rotor, the second ball bearing assembly securely receiving and supporting a proximal portion of the shaft assembly.

17. The motor assembly of claim 16, wherein the first ball bearing assembly and the second ball bearing assembly include the lubricant, such that during operation, the first ball bearing assembly and the second ball bearing assembly are at least partially self-lubricating.

18. The motor assembly of claim 11, further comprising a shaft assembly that extends through a center portion of the rotor, and wherein the shaft assembly is at least partially filled with the lubricant.

19. The motor assembly of claim 18, further comprising a catheter body that houses a drive cable of the shaft assembly, and wherein the catheter body is at least partially filled with the lubricant.

20. A method for manufacturing a catheter pump system, the method comprising:
providing a rotor, the rotor configured to couple to a proximal portion of an output shaft;
coupling a stator at least partially about the rotor, the stator and the rotor defining a radial gap therebetween;
coupling a rotor chamber at least partially between the stator and the rotor within the radial gap, the rotor chamber at least partially enclosing the rotor; and
filling the rotor chamber with a lubricant to prime the catheter pump system with the lubricant prior to operation.
